(19) United States
(12) Patent Application Publication
(10) Pub. No.: US 2024/0170164 A1
(43) Pub. Date: May 23, 2024

(54) METHOD FOR PROVIDING VISUALIZED CLINICAL INFORMATION AND ELECTRONIC DEVICE

(71) Applicant: AESOP Technology Inc., San Francisco, CA (US)

(72) Inventors: Yu-Chuan Li, New Taipei City (TW); An Jim Long, Taipei City (TW)

(73) Assignee: AESOP Technology Inc., San Francisco, CA (US)

(21) Appl. No.: 18/282,795

(22) PCT Filed: Sep. 6, 2021
(86) PCT No.: PCT/US2021/049188

§ 371 Date: Sep. 19, 2023

Related U.S. Application Data
(60) Provisional application No. 63/196,186, filed on Jun. 2, 2021.

(51) Int. Cl. G16H 70/40; G16H 15/00

(52) U.S. Cl. CPC G16H 70/40; G16H 15/00

(57) ABSTRACT

The disclosure provides a method for providing visualized clinical information and an electronic device. The method includes: obtaining a diagnosis result and a treatment means of a patient; creating a clinical information interface based on the diagnosis result and treatment means, wherein the clinical information interface includes a diagnosis result block corresponding to the diagnosis result and a treatment means block corresponding to the treatment means; determining whether a first predetermined correlation exists between the first diagnosis result and the first treatment means; in response to determining that the predetermined correlation exists between the diagnosis result and the treatment means, establishing a connection line between the diagnosis result block and the treatment means block; and in response to determining that no predetermined correlation exists between the diagnosis result and the treatment means, not establishing the connection line between the diagnosis result block and the treatment means block.

### CROSS-REFERENCE TO RELATED APPLICATION

This application claims the priority benefit of U.S. provisional application Ser. No. 63/196,186, filed on Jun. 2, 2021. The entirety of the above-mentioned patent application is hereby incorporated by reference herein and made a part of this specification.

### BACKGROUND

### 1. Technical Field

The disclosure relates to a technology for providing visualized information, and in particular to a method for providing visualized clinical information and an electronic device.

### 2. Description of Related Art

To assist clinical staff in making correct clinical decisions, hospitals often provide relevant clinical alert systems as one of the clinical decision support systems. In most cases, although clinical prescriptions are often characterized by high volume, omissions, and error, clinical staff can make corrections and improvements based on the information provided by the clinical alert system.

In the existing technology, most of the traditional clinical alert systems describe the items to be corrected and improved in words. Moreover, the increased coverage of the clinical alert system has made it necessary to list multiple clinical alerts in a single prescription before corrections and improvements can be made. However, too much writing may impose additional burden on clinical staff with heavy clinical workloads due to the difficulty of referencing.

### SUMMARY

In view of this, the disclosure provides a method for providing visualized clinical information and electronic device, which can solve the above technical problems.

The disclosure provides a method for providing visualized clinical information suitable for an electronic device, which includes: obtaining a first diagnosis result of a patient in a consultation session and obtaining a first treatment means associated with the patient; creating a clinical information interface based on the first diagnosis result and the first treatment means, where the clinical information interface includes a first diagnosis result block corresponding to the first diagnosis result and a first treatment means block corresponding to the first treatment means; determining whether a first predetermined correlation exists between the first diagnosis result and the first treatment means; in response to determining that the first predetermined correlation exists between the first diagnosis result and the first treatment means, establishing a connection line between the first diagnosis result block and the first treatment means block; and in response to determining that no first predetermined correlation exists between the first diagnosis result and the first treatment means, not establishing a connection line between the first diagnosis result block and the first treatment means block.

The disclosure provides an electronic device, which includes a storage circuit and a processor. The storage circuit stores a program code. The processor is coupled to the storage circuit and accesses the program code to execute: obtaining a first diagnosis result of a patient in a consultation session and obtaining a first treatment means associated with the patient; creating a clinical information interface based on the first diagnosis result and the first treatment means, where the clinical information interface includes a first diagnosis result block corresponding to the first diagnosis result and a first treatment means block corresponding to the first treatment means; determining whether a first predetermined correlation exists between the first diagnosis result and the first treatment means; in response to determining that the first predetermined correlation exists between the first diagnosis result and the first treatment means, establishing a connection line between the first diagnosis result block and the first treatment means block; and in response to determining that no first predetermined correlation exists between the first diagnosis result and the first treatment means, not establishing a connection line between the first diagnosis result block and the first treatment means block.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1 is a schematic diagram of an electronic device according to an embodiment of the disclosure.

FIG. 2 is a flowchart of a method for providing visualized clinical information according to an embodiment of the disclosure.

FIG. 3 is a schematic diagram of a clinical information interface according to an embodiment of the disclosure.

FIG. 4 is a schematic diagram of an editing interface according to an embodiment of the disclosure.

FIG. 5 is a schematic diagram of a clinical information interface according to an embodiment of the disclosure.

### DESCRIPTION OF THE EMBODIMENTS

Reference will now be made in detail to the exemplary embodiments of the disclosure, examples of which are illustrated in the accompanying drawings. Wherever possible, the same reference numbers are used in the drawings and the description to refer to the same or like parts.

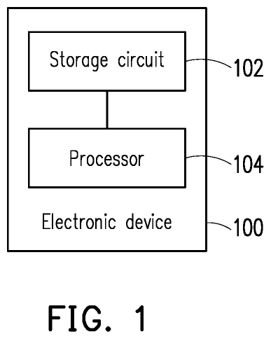

Please refer to FIG. 1, which is a schematic diagram of an electronic device according to an embodiment of the disclosure. In different embodiments, an electronic device 100 is, for example, various computer devices and/or intelligent devices. In some embodiments, the electronic device 100 is, for example, a computer device installed in a medical place and capable of running a hospital information system for doctors or related clinicians to search for the required clinical information. In some embodiments, the electronic device 100 may run a clinical alert system for providing clinical alert information for doctors or related clinicians to correct or improve the prescribed clinical prescription accordingly, but the disclosure is not limited thereto.

In FIG. 1, the electronic device 100 may include a storage circuit 102 and a processor 104. The storage circuit 102 is, for example, any type of fixed or removable random access memory (RAM), read-only memory (ROM), flash memory, hard disk, or other similar devices or a combination of these devices, so as to be configured to record multiple program codes or modules.

The processor 104 is coupled to the storage circuit 102, and may be a general purpose processor, a special purpose processor, a conventional processor, a digital signal processor, multiple microprocessors, one or more microprocessors combined with a digital signal processor core, a controller, a microcontroller, an application specific integrated circuit, ASIC), a field programmable gate array (FPGA), any other kind of integrated circuit, state machine, processor based on advanced RISC machine (ARM), and the like.

In an embodiment of the disclosure, the processor 104 may access the modules and program codes recorded in the storage circuit 102 to implement the method for providing visualized clinical information provided by the disclosure, the details of which are as follows.

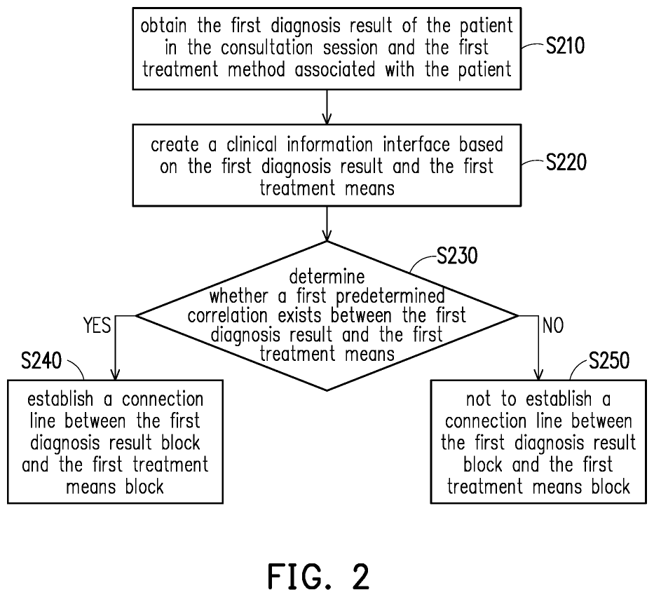

Please refer to FIG. 2, which is a flowchart of a method for providing visualized clinical information according to an embodiment of the disclosure. The method of the present embodiment may be executed by the electronic device 100 of FIG. 1, and the details of each step in FIG. 2 will be described below in accompanying with the components shown in FIG. 1.

In some embodiments, when a clinician (e.g., a physician) conducts a consultation with a patient A (hereinafter referred to as a consultation session), the clinician may input one or more diagnosis results related to the patient A and one or more treatment means that the clinician thinks may be used to deal with these diagnosis results by operating the electronic device 100. In different embodiments, the diagnosis result is, for example, some diseases and/or symptoms, and the treatment means is, for example, some drugs (and the doses thereof) and/or surgery, but the disclosure is not limited thereto.

In one embodiment, the electronic device 100 may include a display (which may be a touch screen), and the processor 104 may control the display to display a user interface, in which the clinician may input the above diagnosis results and treatment means. In some embodiments, the user interface may provide a search box for clinicians to input keywords of diseases/symptoms/drugs/surgeries to be searched. In some embodiments, based on the above keywords, the processor 104 may find out the matched diseases/symptoms/drugs/surgery options for clinicians to select as the diagnosis results and/or treatment means associated with the above consultation sessions, but the disclosure is not limited thereto.

In some embodiments, after the clinician decides the diagnosis result and treatment means of the patient A, the clinician may, for example, trigger a finish button in the user interface. Afterwards, the electronic device 100 may execute the steps in FIG. 2 accordingly. In other embodiments, the electronic device 100 may also execute the steps of FIG. 2 when the clinician selects any diagnosis result and treatment means, but the disclosure is not limited thereto.

For better understanding, it is assumed that the clinician inputs a first diagnosis result and a first treatment means corresponding to the consultation session in the user interface, but this is for example only, and is not intended to limit the possible embodiments of the disclosure.

Accordingly, in step S210, the processor 104 may obtain the first diagnosis result of the patient A in the above consultation session and obtain the first treatment means associated with the patient A.

Thereafter, in step S220, the processor 104 may create a clinical information interface based on the first diagnosis result and the first treatment means, wherein the clinical information interface may include a first diagnosis result block corresponding to the first diagnosis result and a first treatment means block corresponding to the first treatment means.

Next, in step S230, the processor 104 may determine whether a first predetermined correlation exists between the first diagnosis result and the first treatment means. In an embodiment, the processor 104 may determine whether the first treatment means is capable of treating the first diagnosis result. If yes, the processor 104 may determine that the first predetermined correlation exists between the first diagnosis result and the first treatment means, otherwise, the processor 104 may determine that no first predetermined correlation exists between the first diagnosis result and the first treatment means.

For example, suppose that the first diagnosis result and the first treatment means decided by the clinician for the patient A are diabetes and insulin, respectively. In this case, the processor 104 may determine that the first predetermined correlation exists between the first diagnosis result and the first treatment means based on determining that insulin has the ability to deal with (e.g., treat) diabetes. For another example, suppose that the first diagnosis result and the first treatment means decided by the clinician for the patient A are diabetes and sedative respectively. In this case, the processor 104 may determine that no first predetermined correlation exists between the first diagnosis result and the first treatment means based on determining that the sedative does not have the ability to deal with (e.g., treat) diabetes.

In an embodiment of the disclosure, whether the first predetermined correlation exists between a certain diagnosis result and a certain treatment means may also be established in advance by the developer of the above-mentioned clinical alert system according to the existing medical knowledge. For example, since insulin may be used to treat diabetes, the developer may define diabetes and insulin as having the first predetermined correlation with each other in advance. For another example, since sedative may be used to treat insomnia, the developer may define insomnia and sedative as having the first predetermined correlation with each other in advance. For yet another example, because the stool softener may be used to treat constipation, the developer may define constipation and the stool softener as having the first predetermined correlation with each other in advance.

In other embodiments, whether a first predetermined correlation exists between a certain diagnosis result and a certain treatment means may also be based on the technical means described in Taiwan Patent Application No. 110124537, the entire text of which is incorporated herein by reference.

In this case, when the processor 104 determines that no first predetermined correlation has been established between a certain diagnosis result and a certain treatment means in advance, the processor 104 may correspondingly determine that no first predetermined correlation exists between the two, but the disclosure is not limited thereto.

In an embodiment, in response to the processor 104 determining that the first predetermined correlation exists between the first diagnosis result and the first treatment means in step S230, the processor 104 may execute step S240 to establish a connection line between the first diagnosis result block and the first treatment means block.

On the other hand, in response to the processor 104 determining that no first predetermined correlation exists between the first diagnosis result and the first treatment means in step S230, the processor 104 may execute step S250 to not establish a connection line between the first diagnosis result block and the first treatment means block.

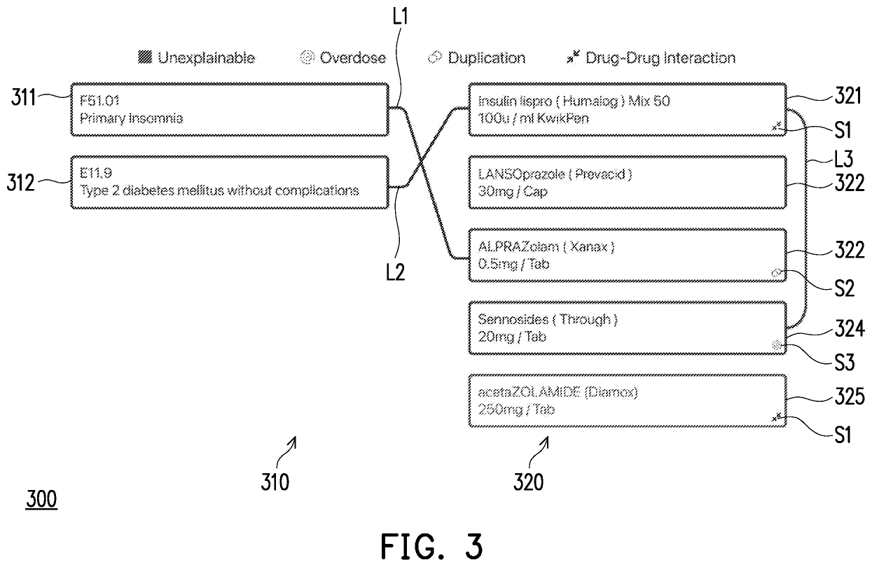

Therefore, the clinicians may more conveniently determine whether the first diagnosis result and/or the first treatment means need to be corrected based on whether a connection line exists between the first diagnosis result block and the first treatment means block. In order to make the above concepts easier to understand, further discussions would be provided in the following with the FIG. 3.

Referring to FIG. 3, which is a schematic diagram of a clinical information interface according to an embodiment of the disclosure. In the scenario of FIG. 3, it is assumed that the diagnosis results and treatment means provided by the clinician for the patient A in the consultation session are illustrated in the following Table 1.

TABLE 1

| Diagnosis results | Treatment means |
|---|---|
| Primary insomnia | Insulin lispro (Humalog) Mix 50 |
| | (unit dose: 100 u/ml Kwikpen) |
| Type 2 diabetes mellitus | LANSOprazole (Prevacid) |
| without complications | (unit dose: 30 mg/Cap) |
| | ALPRAZolam(Xanax) |
| | (unit dose: 0.5 mg/Tab) |
| | Sennosides(Through) |
| | (unit dose: 20 mg/Tab) |
| | acetaZOLAMIDE(Diamox) |
| | (unit dose: 250 mg/Tab) |

In this case, the processor 104 may create a clinical information interface 300 shown in FIG. 3 based on the contents of Table 1. As shown in FIG. 3, the clinical information interface 300 may include diagnosis result blocks 311 and 312 corresponding to the diagnosis results in Table 1 and treatment means blocks 321 to 325 corresponding to the treatment means in Table 1.

In FIG. 3, the clinical information interface 300 may include a first display area 310 and a second display area 320, wherein the first display area 310 may be located on a first side (such as left side) of the clinical information interface 300, and the second display area 320 may be located on a second side (such as right side) of the clinical information interface 300, but the disclosure is not limited thereto.

As shown in FIG. 3, the diagnosis result blocks 311 and 312 may be displayed in the first display area 310, and the treatment means blocks 321 to 325 may be located in the second display area 320.

In the scenario of FIG. 3, since “ALPRAZolam” (which is a sedative) may be used to treat “Primary insomnia” (i.e., insomnia), the processor 104 may establish a connection line L1 in the corresponding diagnosis result block 311 and the treatment means block 323 because the first predetermined correlation exists between “ALPRAZolam” and “Primary insomnia”. Moreover, since “Insulin lispro” (i.e., insulin) may be used to treat “type 2 diabetes mellitus without complications” (i.e., diabetes), the processor 104 may establish a connection line L2 in the corresponding diagnosis result block 312 and the treatment means block 321 because the first predetermined correlation exists between “Insulin lispro” and “type 2 diabetes mellitus without complications”.

In other embodiments, if the clinician provides other treatment means having the first predetermined correlation with “Primary insomnia” (such as drugs that may be used to treat insomnia), then, the processor 104 may establish a treatment means block corresponding to the treatment means in the clinical information interface 300 and establish a connection line between this treatment means block and the diagnosis result block 311, but the disclosure is not limited thereto.

In addition, in other embodiments, if the clinician provides other diagnosis results having the first predetermined correlation with “Insulin lispro”, the processor 104 may establish diagnosis result blocks corresponding to these diagnosis results in the clinical information interface 300 and establish connection lines between the treatment means block 321 and each of these diagnosis result blocks, but the disclosure is not limited thereto.

In an embodiment, if the clinician provides a second treatment means associated with the patient A in the consultation session, the processor 104 may determine whether a second predetermined correlation exists between the first treatment means and the second treatment means. If so, the processor 104 may establish a connection line between the first treatment means block and the second treatment means block after showing the second treatment means block corresponding to the second treatment means in the clinical information interface 300.

In some embodiments, the processor 104 may determine whether the first treatment means and the second treatment means belong to a same preset treatment means combination. If yes, the processor 104 may determine that the second predetermined correlation exists between the first treatment means and the second treatment means, otherwise, the processor 104 may determine that no second predetermined correlation exists between the first treatment means and the second treatment means.

In an embodiment of the disclosure, whether two treatment means belong to the same preset treatment means combination may also be established in advance by the developer of the clinical alert system according to the existing medical knowledge. For example, because the use of insulin may lead to constipation, a stool softener is often prescribed together with insulin. In this case, insulin and stool softeners may be defined as belonging to the same preset treatment means combination. Accordingly, the processor 104 may determine that the second predetermined correlation exists between insulin and the stool softener.

In other embodiments, according to the existing medical knowledge, the developer may define in advance some treatment means that are often prescribed/used together as belonging to the same preset treatment means combination, but the disclosure is not limited thereto.

In the scenario of FIG. 3, the second predetermined correlation exists between “Insulin lispro” and “Sennosides” (i.e., stool softener) because a diabetic patient often has gastrointestinal symptoms due to hyperglycemia, so the processor 104 may establish a connection line L3 between the treatment means blocks 321 and 324 according to the above.

In some embodiments, when the first treatment means and the second treatment means respectively correspond to a first drug and a second drug, the processor 104 may also determine whether a drug-drug interaction exists between the first drug and the second drug. If yes, the processor 104 may mark an interaction indicator in the first treatment means block and the second treatment means block, otherwise, the processor 104 may not mark an interaction indicator in the first treatment means block and the second treatment means block.

In an embodiment of the disclosure, whether a drug-drug interaction exists between two drugs may also be established in advance by the developer of the clinical alert system according to the existing medical knowledge. For example, according to current medical knowledge, there is often drug-drug interaction between antacids and diuretics. Therefore, in the scenario of FIG. 3, the processor 104 may mark an interaction indicator S1 in both the treatment means block 322 corresponding to “Insulin” (i.e., insulin) and the treatment means block 325 corresponding to “acetaZOLAMIDE” (i.e., diuretic).

In an embodiment, when the first treatment means is determined to be the first drug, the processor 104 may determine whether the patient A is currently receiving the treatment of first drug in response to other consultation sessions. If so, the processor 104 may mark a duplicated medication indicator in the first treatment means block, otherwise, the processor 104 may not mark a duplicated medication indicator in the first treatment means block.

In some embodiments, the processor 104 may access the database of the Health Insurance Bureau or other similar institutions to obtain the drug treatment content received by the patient A in other consultation sessions, but the disclosure is not limited thereto.

In the scenario of FIG. 3, assuming that the patient A is taking “ALPRAZolam” in response to other consultation sessions, the processor 104 may mark a duplicated medication indicator S2 in the treatment means block 323 accordingly.

In an embodiment, when the first treatment means is decided to be a first drug having a first dose, the processor 104 may determine whether the first dose of the first drug falls within a preset dose range of the first drug. If not, the processor 104 may mark a dose abnormality indicator in the first treatment means block, otherwise, the processor 104 may not mark a dose abnormality indicator in the first treatment means block.

In an embodiment of the disclosure, the preset dose range (which may be understood as the normal dose range) corresponding to a certain drug may also be established in advance by the developer of the clinical alert system according to the existing medical knowledge. For example, according to existing medical knowledge, the preset dose range corresponding to “Sennosides” is, for example, 3-48 mg per day. In the scenario of FIG. 3, it is assumed that the daily dose of “Sennosides” prescribed by the clinician for the patient A is 240 mg (which has exceeded 48 mg), so the processor 104 may mark a dose abnormality indicator S3 in the treatment means block 324 corresponding to “Sennosides”. In another embodiment, assuming that the daily dose of “Sennosides” prescribed by the clinician for the patient A is less than 3 mg, the processor 104 may also mark a dose abnormality indicator S3 in the treatment means block 324 corresponding to “Sennosides” because the dose does not fall within the preset dose range, but the disclosure is not limited thereto.

For another example, according to the existing medical knowledge, the preset dose range corresponding to “ALPRAZolam” is, for example, 0.5-10 mg per day. Therefore, in other embodiments, assuming that the daily dose of “ALPRAZolam” prescribed by the clinician for the patient A is more than 10 mg, the processor 104 may also mark the dose abnormality indicator S3 in the treatment means block 323 corresponding to “ALPRAZolam”. In another embodiment, assuming that the daily dose of “ALPRAZolam” prescribed by the clinician for the patient A is less than 0.5 mg, the processor 104 may also mark the dose abnormality indicator S3 in the treatment means block 323 corresponding to “ALPRAZolam” because the dose does not fall within the preset dose range, but the disclosure is not limited thereto.

In an embodiment, when the first treatment means is determined to be the first drug and no connection line is connected to the first treatment means block, the processor 104 may determine whether the first drug belongs to a low alert medication (LAM).

In an embodiment of the disclosure, whether a drug is a low alert medication may also be established in advance by the developer of the clinical alert system according to the existing medical knowledge. For example, according to current medical knowledge, even a high dose of “LANSOprazole” may not cause significant adverse effects on human body. Therefore, “LANSOprazole” may be defined as a low alert medication. In other embodiments, the developer may define some drugs with the above characteristics as low alert medications in advance according to the existing medical knowledge, but the disclosure is not limited thereto.

On the other hand, for some drugs (such as “acetaZOLAMIDE”) which are more likely to cause problems to human body due to improper dosage, these drugs may be defined as not low alert medications, but the disclosure is not limited thereto.

In an embodiment, in response to determining that the first drug is not a low alert medication, the processor 104 may adjust the first treatment means block to an alert state (such as marking the outer frame of the first treatment means block in red), otherwise, the first treatment means block may not be adjusted to an alert state.

In the scenario of FIG. 3, although the treatment means block 322 corresponding to “LANSOprazole” (i.e., antacid) is not connected with any connection lines, the processor 104 may not adjust the treatment means block 322 to an alert state because “LANSOprazole” is a low alert medication. On the other hand, because the treatment means block 325 corresponding to “acetaZOLAMIDE” (i.e., diuretic) is not connected with any connection lines and “acetaZOLAMIDE” is not a low alert medication, the processor 104 may adjust the treatment means block 325 to an alert state (such as marking the outer frame of the treatment means block 325 in red).

From another point of view, when the treatment means block 325 is not connected with any connection lines, it means that its corresponding treatment means (i.e., “acetaZOLAMIDE”) is neither used to treat the diagnosis results nor related to other treatment means. That is, there may be doubts about this treatment means. Therefore, the processor 104 may adjust the treatment means block 325 to the alert state to appropriately remind the clinician, but the disclosure is not limited thereto.

It may be seen from the above that the embodiments of the disclosure can not only visually provide alerts to clinicians through connection lines between blocks, but also provide more diversified alerts to clinicians based on various indicator/alert states, thereby effectively improving the convenience for clinicians to review clinical alerts.

In some embodiments, for each block in the clinical information interface 300, clinicians may further edit the blocks by triggering (e.g., clicking) the required blocks.

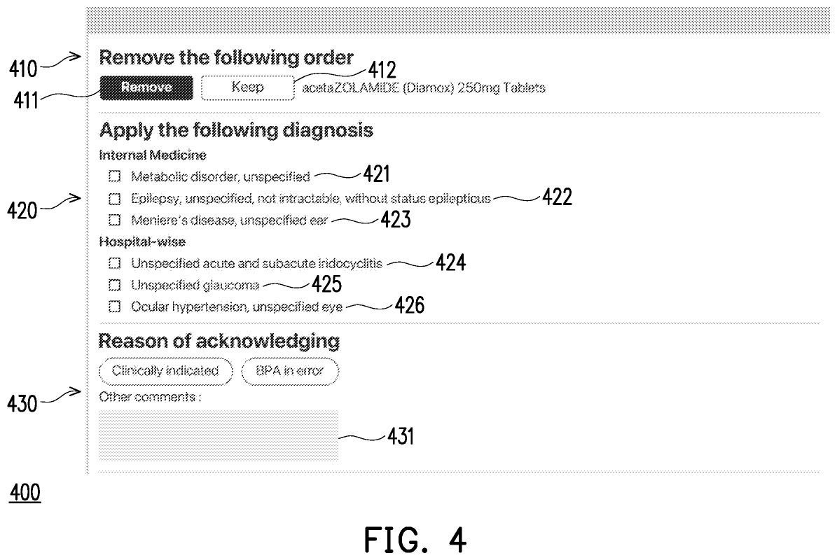

For convenience of explanation, it is assumed that the clinician triggers the treatment means block 325 in the alert state, but the disclosure is not limited thereto. In an embodiment, the processor 104 may switch the display to display the contents shown in FIG. 4 in response to the triggering of the treatment means block 325 by the clinician.

Referring to FIG. 4, which is a schematic diagram illustrating an editing interface according to an embodiment of the disclosure. In FIG. 4, an editing interface 400 may include a first area 410, a second area 420, and a third area 430.

In an embodiment, the first area 410 may include a remove button 411 and a keep button 412, and the clinician may trigger the remove button 411 or the keep button 412 according to whether to keep/remove the treatment means block 325 in the clinical information interface 300. For example, assuming that the clinician determines that there is no problem with the existence of “acetaZOLAMIDE”, the clinician may trigger the keep button 412 accordingly, and otherwise trigger the remove button 411. In one embodiment, in response to determining that the remove button 411 is triggered, the processor 104 may remove the treatment means block 325 from the clinical information interface 300. On the other hand, in response to determining that the keep button 412 is triggered, the processor 104 may keep the treatment means block 325 in the clinical information interface 300, but the disclosure is not limited thereto.

In an embodiment, the second area 420 may include multiple candidate diagnosis results 421-426 corresponding to the treatment means of “acetaZOLAMIDE”, wherein each of the candidate diagnosis results 421-426 may have the first predetermined correlation with the treatment means of “acetaZOLAMIDE”. That is, “acetaZOLAMIDE” may be used to treat diseases/symptoms in each of the candidate diagnosis results 421-426.

In an embodiment, assuming that the clinician belongs to a certain department of a hospital, the candidate diagnosis results 421-423 are, for example, diseases/symptoms often treated by the staff of this department using “acetaZOLAMIDE”, and the candidate diagnosis results 424-426 are diseases/symptoms often treated by the hospital using “acetaZOLAMIDE”, for reference and selection by the clinicians, but the disclosure is not limited thereto.

In one embodiment, in response to determining that any one of the candidate diagnosis results 421-426 is selected (hereinafter referred to as the first candidate diagnosis result), the processor 104 may add a specific diagnosis result block corresponding to the first candidate diagnosis result in the clinical information interface 300. For example, assuming that a clinician selects the candidate diagnosis result 425 (i.e., glaucoma), the processor 104 may add a specific diagnosis result block corresponding to the candidate diagnosis result 425 in the clinical information interface 300, and establish a connection line between the specific diagnosis result block and the treatment means block 425, but the disclosure is not limited thereto.

In an embodiment, the third area 430 may include a dialog box 431, which may be used for clinicians to input the reason for using the treatment means of “acetaZOLAMIDE”. For example, when the clinician thinks that there is nothing wrong with using “acetaZOLAMIDE” treatment means for the patient A in the consultation session, the clinician may enter the relevant reasons for use in the dialog box 431 to explain the reasons for using “acetaZOLAMIDE”, but the disclosure is not limited thereto.

In other embodiments, when the clinician clicks on other blocks in the clinical information interface 300 (such as the treatment means blocks 321-324), the processor 104 may also show corresponding editing interfaces for further editing by the clinician, but the disclosure is not limited thereto.

Accordingly, the clinicians may further edit/explain the contents of the clinical information interface 300 through the editing interface 400, thereby providing better operation flexibility.

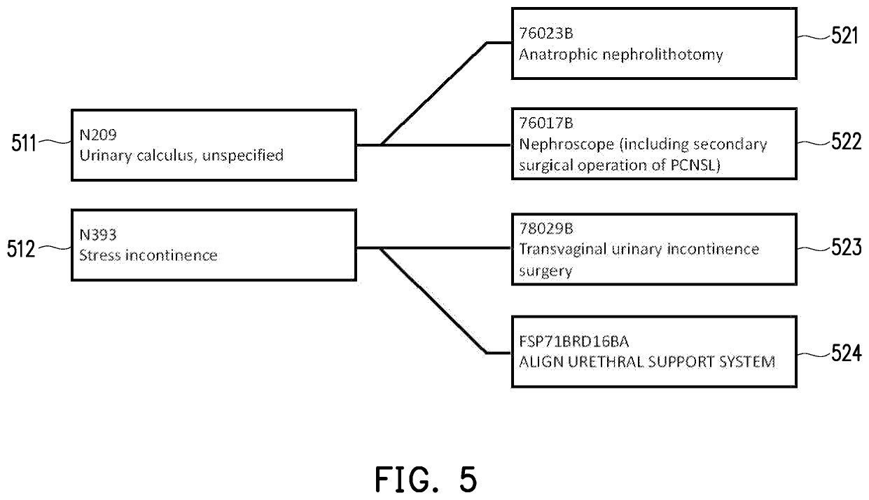

Referring to FIG. 5, which is a schematic diagram of a clinical information interface according to an embodiment of the disclosure. In the present embodiment, it is assumed that the diagnosis result and treatment means determined by the clinician for a patient B are illustrated in the following Table 2.

TABLE 2

| Diagnosis results | Treatment means |
|---|---|
| Urinary calculus, | Anatrophic nephrolithotomy |
| unspecified | Nephroscope (including secondary |
| | surgical operation of PCNSL) |
| Stress incontinence | Transvaginal urinary incontinence surgery |
| | ALIGN URETHRAL SUPPORT SYSTEM |

In this case, the processor 104 may create a clinical information interface 500 shown in FIG. 5 based on the contents of Table 2. As shown in FIG. 5, the clinical information interface 500 may include diagnosis result blocks 511 and 512 corresponding to the diagnosis results in Table 2 and treatment means blocks 521 to 524 corresponding to the treatment means in Table 2.

In the scenario of FIG. 5, assuming that “uric calculus, unspecified” has the first predetermined correlation with both “anatrophic nephrolithotomy” and “nephroscope (including secondary surgical operation of PCNSL)”, then the processor 104 may establish a connection line between the diagnosis result block 511 and the treatment means block 521 and establish a connection line between the diagnosis result block 511 and the treatment means block 522.

Moreover, assuming that “stress incontinence” has the first predetermined correlation with both “transvaginal urinary incontinence surgery” and “ALIGN URETHRAL SUPPORT SYSTEM”, then the processor 104 may establish a connection line between the diagnosis result block 512 and the treatment means block 523 and establish a connection line between the diagnosis result block 512 and the treatment means block 524.

Accordingly, the clinician may also conveniently know the relevant clinical alerts based on the clinical information interface 500, and then make necessary adjustments and improvements.

To sum up, the embodiment of the disclosure enables integration of multiple clinical alerts in a visual way, and allows clinicians to conveniently see whether the determined diagnosis results and treatment means need to be corrected and improved through connection lines, blocks, colors and various indicators. Therefore, the operation burden of clinician may be effectively reduced.

It will be apparent to those skilled in the art that various modifications and variations may be made to the structure of the disclosure without departing from the scope or spirit of the disclosure. In view of the foregoing, it is intended that the disclosure cover modifications and variations of this disclosure provided they fall within the scope of the following claims and their equivalents.

To the claims:

1. A method for providing visualized clinical information, adapted to an electronic device, the method comprising:
obtaining, via a user interface, a first diagnosis result and a first treatment means inputted to the user interface;
creating a clinical information interface based on the first diagnosis result and the first treatment means, wherein the clinical information interface comprises a first diagnosis result block corresponding to the first diagnosis result and a first treatment means block corresponding to the first treatment means;
determining whether a first predetermined correlation exists between the first diagnosis result and the first treatment means;
in response to determining that the first predetermined correlation exists between the first diagnosis result and the first treatment means, establishing a connection line between the first diagnosis result block and the first treatment means block; and
in response to determining that no first predetermined correlation exists between the first diagnosis result and the first treatment means, not establishing the connection line between the first diagnosis result block and the first treatment means block.

2. The method of claim 1, wherein the clinical information interface comprises a first display area and a second display area, wherein the first diagnosis result block is displayed in the first display area and the first treatment means block is displayed in the second display area.

3. The method of claim 2, wherein the first display area is located at a first side of the clinical information interface, the second display area is located at a second side of the clinical information interface, and the first side is opposite to the second side.

4. The method of claim 1, further comprising:
obtaining a second diagnosis result and showing a second diagnosis result block corresponding to the second diagnosis result in the clinical information interface; and
in response to determining that the first predetermined correlation exists between the second diagnosis result and the first treatment means, establishing the connection line between the second diagnosis result block and the first treatment means block.

5. The method of claim 1, further comprising:
obtaining a second treatment means and showing a second treatment means block corresponding to the second treatment means in the clinical information interface; and
in response to determining that the first predetermined correlation exists between the first diagnosis result and the second treatment means, establishing the connection line between the first diagnosis result block and the second treatment means block.

6. The method of claim 1, further comprising:
in response to determining that the first treatment means is capable of treating the first diagnosis result, determining that the first predetermined correlation exists between the first diagnosis result and the first treatment means; and
in response to determining that the first treatment means is incapable of treating the first diagnosis result, determining that no first predetermined correlation exists between the first diagnosis result and the first treatment means.

7. The method of claim 1, further comprising:
obtaining a second treatment means and showing a second treatment means block corresponding to the second treatment means in the clinical information interface; and
in response to determining that a second predetermined correlation exists between the first treatment means and the second treatment means, establishing the connection line between the first treatment means block and the second treatment means block.

8. The method of claim 7, further comprising:
in response to determining that the first treatment means and the second treatment means belong to a same preset treatment means combination, determining that the second predetermined correlation exists between the first treatment means and the second treatment means; and
in response to determining that the first treatment means and the second treatment means do not belong to any same preset treatment means combination, determining that no second predetermined correlation exists between the first treatment means and the second treatment means.

9. The method of claim 7, wherein the first treatment means comprises a first drug, the second treatment means comprises a second drug, and the method further comprises:
in response to determining that a drug-drug interaction exists between the first drug and the second drug, marking an interaction indicator in the first treatment means block and the second treatment means block.

10. The method of claim 1, wherein the first treatment means comprises a first drug and a first dose of the first drug, and the method further comprises:
in response to determining that the first dose of the first drug does not fall within a preset dose range of the first drug, marking a dose abnormality indicator in the first treatment means block.

11. The method of claim 1, wherein the first treatment means corresponds to a patient and comprises a first drug and a first dose of the first drug, and the method further comprises:
in response to determining that the patient is currently receiving the first drug in response to other consultation sessions, marking a duplicated medication indicator in the first treatment means block.

12. The method of claim 1, wherein the first treatment means comprises a first drug, and the method further comprises:
in response to determining that the first treatment means block is not connected with any connection line, determining whether the first drug belongs to a low alert medication;
in response to determining that the first drug does not belong to the low alert medication, adjusting the first treatment means block to an alert state; and
in response to determining that the first drug belongs to the low alert medication, not adjusting the first treatment means block to the alert state.

13. The method of claim 1, further comprising:
in response to determining that the first treatment means block is triggered, displaying an editing interface corresponding to the first treatment means, wherein the editing interface comprises a first area, and the first area comprises a remove button and a keep button;
in response to determining that the remove button is triggered, removing the first treatment means block from the clinical information interface; and
in response to determining that the keep button is triggered, keeping the first treatment means block in the clinical information interface.

14. The method of claim 13, wherein the editing interface further comprises a second area, wherein the second area comprises a plurality of candidate diagnosis results corresponding to the first treatment means, and each of the candidate diagnosis results has the first predetermined correlation with the first treatment means, and the method further comprises:
in response to determining that a first candidate diagnosis result in the candidate diagnosis results is selected, adding a specific diagnosis result block corresponding to the first candidate diagnosis result in the clinical information interface, and establishing the connection line between the specific diagnosis result block and the first treatment means block.

15. The method of claim 13, wherein the editing interface further comprises a third area, wherein the third area comprises a dialog box, wherein the dialog box is used for inputting a reason for using the first treatment means.

16. The method of claim 1, wherein the first treatment means comprises a surgery.

17. An electronic device, comprising:
a non-transitory storage circuit, storing a program code;
a processor, coupled to the non-transitory storage circuit and accessing the program code to execute:
obtaining, via a user interface, a first diagnosis result and a first treatment means inputted to the user interface;
creating a clinical information interface based on the first diagnosis result and the first treatment means, wherein the clinical information interface comprises a first diagnosis result block corresponding to the first diagnosis result and a first treatment means block corresponding to the first treatment means;
determining whether a first predetermined correlation exists between the first diagnosis result and the first treatment means;
in response to determining that the first predetermined correlation exists between the first diagnosis result and the first treatment means, establishing a connection line between the first diagnosis result block and the first treatment means block; and
in response to determining that no first predetermined correlation exists between the first diagnosis result and the first treatment means, not establishing the connection line between the first diagnosis result block and the first treatment means block.
